(19) United States
(12) Patent Application Publication
(10) Pub. No.: US 2025/0152967 A1
(43) Pub. Date: May 15, 2025

(54) Method and Apparatus for Wearable Device for Closed-Loop Transcranial Photobiomodulation Stimulation Using Cognitive Testing

(71) Applicant: SENS.AI INC., Whistler (CA)

(72) Inventors: Paola Telfer, Whistler (CA); Corey Julihn, Langley (CA); Estate Sokhadze, Greenville, SC (US)

(21) Appl. No.: 18/840,887

(22) PCT Filed: Feb. 23, 2023
(86) PCT No.: PCT/IB2023/051699

§ 371 Date: Aug. 22, 2024

Related U.S. Application Data
(60) Provisional application No. 63/313,265, filed on Feb. 23, 2022.

(51) Int. Cl. A61N 5/06

(52) U.S. Cl. CPC A61N 5/0622; A61N 2005/0626; A61N 2005/0647; A61N 2005/0659; A61N 2005/0663

(57) ABSTRACT

Devices and methods for stimulation and modulation of brain activity using transcranial photobiomodulation (PBM) or brainwave or heart rate variability (HRV) biofeedback and analyzing and monitoring electroencephalographic (EEG) electrical activity evoked during, before and/or after neurocognitive tests using event-related potential (ERP) and evoked and induced EEG oscillations (ERO) measures in human users. Electroencephalography (EEG) and photobiomodulation (PBM) devices in the form of wearable apparatus with headphones for stimulating brain with PBM or using biofeedback training with concurrent EEG monitoring and evaluation of electrical activity generated by a person's brain during stimulation are described, along with description of methods for testing person's cognitive and physiological state during cognitive tests using the provided devices. PBM light is pulsed to stimulate an increase in targeted brain activity by providing additional energy to mitochondria and activating other photosensitive brain processes. PBM stimulation is combined with EEG sensors for detecting EEG responses during cognitive tests using event-related potentials (ERP) and evoked and induced EEG oscillations (ERO) for evaluation of user's cognitive status and treatment of functional outcomes.

### PRIOR RELATED APPLICATIONS

This application claims the benefit of priority of prior-filed U.S. Provisional Application 63/313,625, filed Feb. 23, 3022.

### FIELD OF THE INVENTION

The present invention relates to devices and methods for stimulating, monitoring, and analyzing electrical activity generated by the brain of a person. Specifically, the invention provides electroencephalography (EEG) and photobiomodulation (PBM) devices for monitoring and stimulating electrical activity generated by a person's brain during cognitive tests. Also described are methods for stimulating a person's cognitive and physiological state using the provided PBM device or biofeedback methods, and assessment of electrocortical responses using EEG and event-related potential (ERP) responses and EEG oscillations (ERO) evoked during executive function tests. The invention provides for the continuous or pulsing of PBM light to stimulate an increase in brain activity and providing additional energy to mitochondria and enhancing cognitive functions assessed with EEG sensors and cognitive tests.

### BACKGROUND OF THE INVENTION

Transcranial neurostimulation is the purposeful modulation of the central nervous system's activity. Traditionally this has been done through transcranial electrical or magnetic stimulation via but not limited to transcranial Alternating Current Stimulation (tES), transcranial Direct Current Stimulation (tDCS), Transcranial Magnetic Stimulation (TMS), or pulsed Electromagnetic Field stimulation (pEMF). Another method of transcranial neuromodulation is photobiomodulation (PBM) that uses non-ionizing photonic energy to create photochemical changes inside cortical cellular structures, usually mitochondria. This technique has primarily been used to reduce pain and inflammation and speed the healing process of damaged tissue.

Photobiomodulation have been shown to photostimulate brain cytochrome c oxidase activity and activate other photosensitive chemical processes in the brain. Current data showed that photons in 600 nm-1,100 nm wavelengths range are absorbed by cytochrome c oxidase and increases the activity of the mitochondrial respiratory chain resulting in increased adenosine triphosphate (ATP) production. Transcranial PBM in near-infrared range also alters nitric oxide (NO) levels, whereas near-infrared PBM modulates reactive oxygen species (ROS) and reactive nitrogen species resulting in increased energy processes in the mitochondria. There is taking place as well modulation of the expression of numerous genes in both the mitochondria and the brain cells (Hamblin, 2016).

Transcranial photobiomodulation (tPBM) has been shown in numerous clinical research studies as a safe, side effects free non-invasive neuromodulation method for the reduction of symptoms in a variety of neurological and psychiatric conditions. Several studies reported cognitive enhancement resulting from tPBM, for instance, Saltmarche et al. (2017) reported significant improvement in cognition in mild to moderately severe dementia cases. De la Torre et al. (2020) review examines the impact of reduced energy supply in the aging brain and provides rationale for tPBM therapy for mild cognitive impairment (MCI) and in several neurological conditions including Parkinson's disease, depression, traumatic brain injury, and stroke. Review describes studies showing that tPBM enhances the metabolism of neurons and results in anti-inflammatory, anti-apoptotic, antioxidant responses, neurogenesis, and synaptogenesis. Application and usability of tPBM is not limited to only neurological and psychiatric conditions. Blanco et al. (2017) applied laser stimulation to the prefrontal cortex and found that prefrontal rule-based learning was substantially improved following transcranial infrared laser stimulation in healthy participants. El Khoury et al. (2019) explored whether tPBM can modulate brain activity in young adults using fMRI (functional magnetic resonance imaging). The fMRI findings indicated that tPBM did have an impact on brain activity, but only when the cortical region was functionally active during performance on a task. Vargas et al. (2017) described beneficial neurocognitive effects of tPBM in older adults during performance in attention (psychomotor vigilance task) and memory (delayed match-to-sample task) tests and recorded EEG and fMRI effects. The EEG and fMRI outcomes showed that tPBM increases resting-state EEG alpha, beta, and gamma bands power, and enhances more pronounced prefrontal BOLD-fMRI response. Wang et al. (2019) used tPBM to modulate EEG measures and demonstrated effects of tPBM on EEG activity by recording EEG before, during, and after the application of continuous wave tPBM to the right forehead of human subjects. The results showed that tPBM increased EEG powers of the alpha (8 to 13 Hz) and beta (13 to 30 Hz) rhythms at several scalp topographies. Berman & Nichols (2019) review usability of application of transcranial PBM combined with EEG biofeedback in treatment of neurodegenerative disorders such as Alzheimer and dementia.

U.S. Pat. No. 10,987,521 B1 by Chicchi describes a system and methodology for treating brain disorders with PBM therapy, with claims that treatment in Parkinson's disease, Alzheimer's disease, concussions, depression, strokes, and other brain diseases or injuries have shown dramatic improvement under such treatment protocols. US 2009/0254154 and US 2010/0204762 by De Taboada discloses an apparatus and method for indicating treatment site locations for phototherapy to the brain. In some embodiments, the apparatus is a headpiece wearable by a patient. There are more patents describing portable wearable tPBM devices, many of them are filed by Lim and include both transcranial and intra-nasal PBM devices (AU 2015388475 B2, WO 2019/053625 A1). Described systems use among other methods also EEG as a technique that measures the brain's electrical activity in delta, theta, alpha, beta, and gamma EEG bands. US 2019/0335551 A1 by Williams et al. described PBM system for many users, including such functions as plurality of physiological monitoring biosensors (EEG, EMG, ECG, etc.).

Most directly relevant are series of patents by Huang (US 2021/0001147, US 2021/0016103 A1, U.S. Pat. No. 10,821,298 B, US 2021/0023391) disclosing method and apparatus for brain function enhancement that describes a system for transcranial photobiostimulation of brain of a subject. Patents presents drawings from a pilot study where changes of EEG bands using spectral analysis are presented during tPBM along with fMRI changes induced by tPBM. In addition, patents describe improvements in reaction time and accuracy in neurocognitive tests such as psychomotor vigilance and delayed match-to-sample test. These patents describe cognitive enhancement after tPBM in older adults, as tPBM increased EEG alpha, beta, and gamma along with results showing that tPBM evoked fMRI responses.

However, the prior art has no evidence of transcranial photobiomodulation utilized with event-related potential (ERP) recorded in cognitive tests including any research studies where effects of transcranial photobiomodulation are investigated either in clinical condition or in any patent disclosures. This represents a significant limitation of the prior art because ERP technique is one of the most informative methods of exploration and monitoring of the stages of information processing in the brain. Measures such as amplitude and latency of selected ERP waves recorded at specific topographies allow analysis of sensory and perception-relation processes, as well as higher-level processing stages including attention, cortical inhibition, memory update, error monitoring and other cognitive activities termed also under executive functions definition (Luck, 2014). ERPs provide a method of investigation of cognitive processes not only in typical individuals, but also provides a sensitive tool to assess differences in patients with neurological and psychiatric conditions. Despite significant advances in functional neuroimaging such as fMRI, ERP-based metrics still represents an important instrument in neurology and psychiatry, since some neuropsychiatric diseases correlate with known alterations in ERP patterns that can serve as valid biological neuromarkers for functional diagnostic or for better understanding of the disturbed cognitive functions in psychiatric and neurological conditions.

There are patents filed that describe a portable wearable device with EEG and event-related potential (ERP) functions, including those that have methods of ERP analysis disclosed and several cognitive tests with ERP recording described. There are among them wearable systems that can analyze and assess a person's brain health by integrating the use of EEG and ERP metrics during cognitive testing. Such systems are able to provide for early detection of neurological and psychiatric disorders such as mild cognitive impairment (MCI), dementia, including Alzheimer's disease, and other dementia-type disorders, as well as brain injury states such as mild traumatic brain injury (mTBI). Some of these patents, e.g., U.S. Pat. No. 9,675,292 B2 and EP 2260760 B1 by Fadem describe ERP systems suitable for clinical use that includes an integrated headset that performs an evoked response (ERP) test. Other patents, i.e., WO2020/223397 A1 by Mcloughlin, describe mental fitness assessment systems in healthy person and claim that EEG and ERP measures are indicative of an emotional or cognitive state of the person; and allow assessment of a mental fitness state of the person based on the electrocortical metrics.

During the normal process of aging, humans may experience a certain amount of age-related cognitive decline (ARCD) resulting in increased difficulty in demanding situations and decreased ability to focus attention under time pressure conditions. Individuals with ARCD experience decline in cognitive functioning resulting in decrement of performance effectiveness in tasks that require attention, short- and long-term memory, fast motor reaction, speeded decision making, as well as processing and comprehension of situational demands. Tests using ERP and EEG oscillations (ERO) are known to be one of the best techniques to evaluate the status of cognitive decline both in elderly and in predisposed younger users or those after diseases or disorders known to be associated with decreased cognitive functioning, such as concussion, TBI, or infectious diseases resulting in post-disease “brain fog”.

Other useful EEG measures in cognitive tests are based on wavelet-based time-frequency analysis of EEG oscillations (ERO) in response to stimuli in cognitive tests. More information about time-frequency wavelet-based analysis of EEG and about EEG evoked and induced gamma oscillations (ERO) can be found in the publication of Tallon-Baudry & Bertrand (1999) that describes evoked and induced EEG gamma oscillations (30-100 Hz with most usable gamma range being in 35-45 Hz range). The review focuses on the literature on gamma oscillatory activities in humans and describes the different types of gamma responses and how to analyze them. Evidence presented by researchers suggests that one particular type of gamma activity, specifically induced gamma oscillations can be observed during the construction of an object representation is discussed. The paper has illustrations of evoked and induced gamma EEG oscillations and explanation of their role.

Neuronal gamma-band oscillations, along with other EEG bands oscillations, can be recorded at different scalp topographies (as well as in cortical and subcortical areas), and can be evoked or induced by different stimuli or tasks, such as, for instance, ERP design tests. Event-related oscillatory activity (ERO) in various frequency bands (e.g., delta, theta, alpha etc.) reflects different aspects and stages of information processing. Alpha oscillatory responses increase with simple working memory tasks and decrease with demanding memory tasks. Beta oscillatory responses are important in attention related tasks and some affective tests, for instance recognition of facial expression in humans. Event-related theta oscillatory responses have been proposed to be related to the memory processes.

Wavelet analysis is useful for single trial analysis of EEG oscillation in rare responses, such as for instant response-locked ERP occurring after committed error in speeded cognitive tasks requiring motor response. Clemans et al. (2012) reported that response-locked ERP used as measures of error processing are the error-related negativity (ERN) and the error-related positivity (Pe) that occur following committed error in speeded reaction time tests can be recorded in a form of low frequency (4-8 Hz) EEG oscillations at the midline frontal and frontocentral EEG sites. Error processing using time-frequency analysis in the form of a wavelet transform is described as an alternative method to isolate a theta waveform in the time-frequency domain and to obtain a single time-frequency correlates of ERN and Pe for each error trial. These results of the study indicate that the suggested alternative single trial time-frequency error analysis method is suitable for detection of error-related processes both in healthy individuals and patients with psychiatric conditions.

Davoudi et al. (2021) describe frequency-amplitude coupling as a new approach for decoding of attention-related processes in cognitive tasks. The method has been described in said patent as reflecting information processing in the brain and references cross-frequency coupling. It is generally assumed that some EEG frequencies demonstrate phase-amplitude coupling processes, for instance theta-gamma phase amplitude coupling plays a crucial role in perception, memory, and attention (Canolty et al., 2006; Koster et al., 2014).

Summary of the state-of-art related to present invention and limitations of current status denoted the needs in art to be addressed. Transcranial photobiomodulation (tPBM) is gaining more attention both in scientific literature and patents judging by increased number of publications with studies that sufficiently well describe neurobiological mechanisms how light in 600-1100 nm range affects the brain and patents disclosing the same. Very detailed and scientifically justified effects of tPBM are available and widely accepted. This positions tPBM as one of the most popular and viable neuromodulation techniques that has no side effects. There are filed patents that describe tPBM effects on brain activity, including several of those that comprises EEG recording and some functional magnetic resonance imaging (fMRI) outcomes of tPBM in healthy subjects and in various neurological and psychiatric population, with more widely accepted target conditions are dementia, mild cognitive impairment (MCI), Alzheimer, Parkinson, stroke, traumatic brain injury (TBI), etc. Furthermore, several patents disclose applications and methods for enhancement of cognitive functions. Some, though only few, combine tPBM with neurofeedback, while several of patents cover effects of tPBM on EEG activity, in particular changes in alpha, beta, and gamma activity, along with improvement in cognitive functions and in performance (i.e., reaction time and accuracy) in cognitive tests. Some of the above devices (tPBM and EEG) are wearable, though only few can be operated with smartphones/tablets.

There are numerous patents that describe apparatus and methods for wearable EEG systems for various indications and multiple embodiments, including those that disclose applications of various event-related potential (ERP) tests. Some of them have detailed description of stimulus-locked ERPs and response-locked error-related negativity (ERN) and error-related positivity (Pe) potentials. There are patents describing application of time-frequency analysis of EEG responses using wavelet transformation and justifying usability of the method for single trial analysis of EEG responses.

The literature has many publications on gamma oscillations in tasks similar or even the same as ERP paradigm, including description of evoked and induced gamma, and literature about theta and gamma and other EEG rhythms phase-amplitude coupling as useful measures of cognitive functions (Lisman & Jensen, 2013; Koster et al., 2014). Evoked and induced gamma oscillations, as well as theta oscillations, and their coupling is an area not covered in patents or publications related to photobiomodulation or any form of biofeedback. There are patents that list, as possible embodiments, the inclusion of other biometrics along with EEG, such as for instance heart rate (HR), heart rate variability (HRV) and other vitals signals. There are many patents and published scientific literature that focus on HRV biofeedback and EEG biofeedback and usability of biofeedback training for various clinical and performance improvement applications (Lehrer & Gevirtz, 2014; Sherlin et al., 2011).

Review of the status of prior art demonstrates that current tPBM systems and methods do not disclose effects of transcranial photobiomodulation on performance in cognitive tests with EEG recording for ERP testing and evoked and induced EEG oscillations in cognitive tests. Availability of such systems are needed to enhance brain stimulation methodology and usability by providing availability to monitor improvements in behavioral, EEG,ERP and heart rate variability (HRV) measures assessed using cognitive tests.

Therefore, there remains a need for new and improved methods and systems for brain function improvement to overcome the limitations stated above. There is a need for wearable transcranial photobiomodulation-based physiological and neurological stimulation devices that provides one or more continuous wave or pulsed light sources, such as lasers and LEDs, to stimulate specific areas of brain and have functions allowing utilization of physiological measures using EEG and HRV for assessment effects of tPBM protocols or enter modification of the tPBM protocol. Furthermore, it is needed in the art for devices that in addition to tPBM are capable to be used for EEG training using either EEG or heart rate variability (HRV) biofeedback with ability to test EEG or HRV biofeedback training outcomes using ERP and evoked EEG oscillations during cognitive tests.

What is needed in the art is a wearable tPBM system and method for administration of a battery of cognitive tests such flanker test, auditory and visual oddball tests, and other executive functions tests that provide information about such processes as focused and sustained attention, cortical inhibition, error monitoring and correction functions that are reflecting changes induced by the tPBM or combination of tPBM with biofeedback training.

More specifically what is needed is a tPBM system and method allowing to synchronize presentation of stimuli in visual modality delivered through controller connected with wearable device and smartphone or tablet or in auditory modality delivered through the headphones with EEG signal recorded with EEG sensors mounted in the wearable device allowing correct timing of stimulation event and EEG recording during each presented stimulus. What is needed in the art is the ability to record not only EEG metrics but also behavioral responses in said cognitive tests administered during or after tPBM or combined tPBM sessions with biofeedback.

What is still further needed in the art is an improved methodology of detection of EEG responses to stimuli during cognitive tests, including ability to recognize and identify EEG oscillations not only with the averaging methods but with ability to analyze EEG signal in single trial mode using EEG oscillations occurring in theta and gamma bands in response to stimuli and processed using time-frequency analytical methods based on wavelet transformation.

What is further needed in the art is a practical and effective device and method for applying tPBM to users' brain and record event-related potentials, evoked and induced EEG oscillations, heart rate variability changes, and behavioral responses during cognitive tests to evaluate reaction time of motor response in a form of pressing a button on a controller, and accuracy of responses assessed using such metrics as number of percentage of total errors, number of incorrect responses including those related to missed response, omission errors, or pressing incorrect button or pressing button when response was not required, thus committing commission error.

What is yet further needed in the art is a device and method for applying tPBM to users' brain to improve outcomes of EEG biofeedback (neurofeedback) or heart rate variability (HRV) biofeedback with capacity to assess results of tPBM on efficacy of neurofeedback or HRV biofeedback training using functional measures of ERP and EEG oscillations (ERO) changes in cognitive tests.

The present invention along with continuous wave stimulation uses pulsed PBM light at targeted frequencies known to provide additional energy to mitochondria at targeted locations.

The invention also combines PBM stimulation with electroencephalography (EEG) and heart rate variability sensors, and experimental system for administering cognitive tests aimed at assessment of attention, working memory, cortical inhibition, performance monitoring and other executive functions using EEG oscillations (ERO) and ERP measures. This allows for a novel joint application tPBM-based neurostimulation protocols with evaluation of a person's performance in cognitive tests using EEG and ERP correlates of executive functions and mental focus state of a person.

### SUMMARY OF THE INVENTION

The present invention provides a wearable head mounted device, with headphones and adjacent cognitive tests controller unit, that incorporates embedded tPBM and EEG and other biometric sensors. Data collected from the sensors provide data patterns that are analyzed during cognitive tests administered before, during, after individual tPBM sessions or following the course of tPBM therapy, EEG biofeedback or heart rate variability (HRV) biofeedback. The biometric data includes but is not limited to: EEG (electroencephalography), heart rate, pulse volume, heart rate variability (HRV), and other physiological measures. The present invention utilizes for biometrics such physiological signals as EEG recorded from locations which may include at Fz, Cz, and Pz and photoplethysmogram (PPG) or pulse oximetry recorded from the ear. Analysis of EEG and physiological biometric data and presentation of stimulation during cognitive tests is processed using a smartphone or tablet, and/or a remotely located computer.

### BRIEF DESCRIPTION OF THE DRAWINGS

The invention will be better understood according to the following detailed description of several embodiments with reference to the attached drawings, in which:

FIG. 1 illustrates one embodiment of a wearable device with headphones for photobiomodulation stimulation with biosensors and biodata processing and monitoring unit and cognitive stimulation controller with buttons to record motor responses;

FIG. 2 illustrates paradigm of arrows flanker test with examples of stimuli in cognitive test;

FIG. 3 illustrates error response-locked error-related negativity (ERN) and error-related positivity (Pe) waveforms;

FIG. 4 illustrates single trial ERN and Pe measures processed using time-frequency wavelet-based transformation;

FIG. 5 illustrates frontal event-related potentials (ERP) in response to target and non-target stimuli in visual cognitive test;

FIG. 6 illustrates evoked and induced EEG gamma oscillations in 35-35 Hz range in response to target and non-target stimuli in visual cognitive test processed using time-frequency wavelet transformation;

FIG. 7 illustrates theta and gamma frequency oscillations and their frequency coupling during a cognitive test.

A more detailed understanding of the disclosed device and method will be obtained from the following description of the embodiments along with the figures, drawings and the claims of the present invention.

### DETAILED DESCRIPTION OF THE INVENTION

Each of the examples of the embodiments of the intention are provided by an explanation of the specifics of the invention, and should not be considered as a limitation of the invention. To those skilled in the art, it is understood that modifications can be made in the present invention within the scope or spirit of the apparatus, system, and methods of the present invention.

Further, in this invention, the terms such as “person”, and “user”, and “wearer”, and “patient”, and “human”, and “individual” and “subject” are used interchangeably to refer to a person using the said invention. “Treatment” or “stimulation” or “therapy” or “training” or “session” or “assessment” or “test” as used herein, covers the treatment of the person to obtain benefits or intended results in the person/user/wearer/patient/human/individual, aimed at analysis of cognitive function or prevention of the decline of cognitive function or providing a short-term or long-term cognitive improvements resulting from above said treatment.

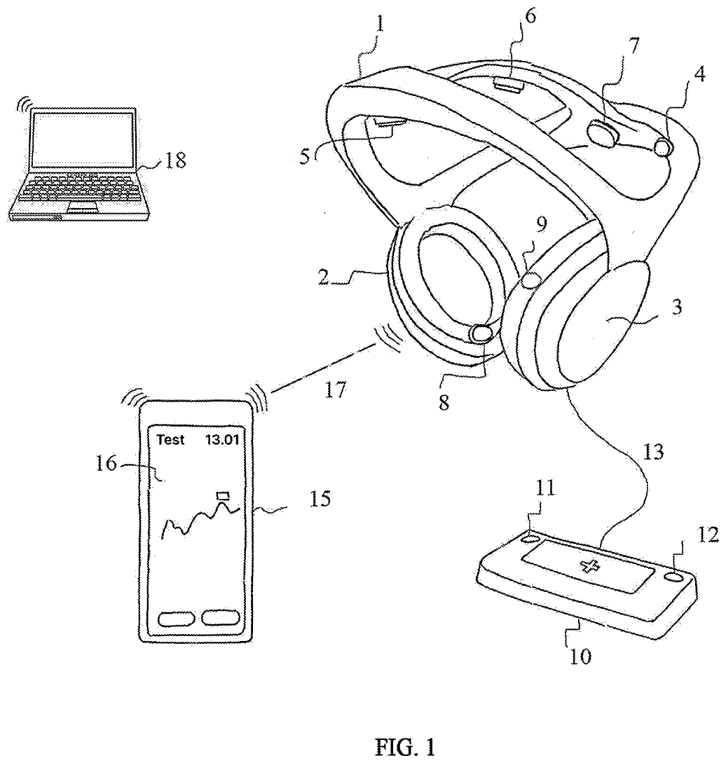

In one aspect, the present invention provides EEG and PPG sensors in a head mounted device 1 with headphones 2 and 3 illustrated in FIG. 1. In an embodiment illustrated in FIG. 1, the headphones of the present invention combine EEG (electroencephalography) sensors 5, 6 and 7 for EEG and event-related potential (ERP) and EEG oscillations (ERO) measurement and phoplethysmography (PPG) sensor 9 for heart rate variability (HRV) measurement in a wearable head mounted device with headphones. In an embodiment, a PPG sensor 9 is incorporated inside an over-ear headphone design which reduces ambient noise allowing for increased accuracy. The present invention provides a wearable head mounted headphone set 1 with embedded biometric sensors that collect physiological signals from the user. The device includes Bluetooth (wireless) audio and data transmission 17 which may be used to connect the device 1 to smartphone/mobile device 15, with graphic touchscreen display 16, and said smartphone/mobile device 15 has wireless wi-fi connection with remotely located computer 18. The device 1 may also include a rechargeable battery, speakers, microphone, and has wire 13 connecting headphones with cognitive test controller 10.

Electrodes are used to collect EEG signals. FIG. 1 illustrates electrodes that are placed at Fz 5, Cz 6, and Pz 7 locations according to the International 10-20 system and include reference and ground electrodes at A1 or A2 or M1 and M2 locations. Photobiomodulation LEDs such as one of them 4 illustrated in FIG. 1 are embedded in head mounted device 1.

Photoplethysmography methods are used to collect additional biometrics for measuring heart rate (HR), HRV, and pulse volume. Photoplethysmography (PPG) is an optical measurement of the absorption of specific wavelengths of light by the body. A PPG sensor containing LEDs and photosensors is placed inside one of the earpieces and positioned against the outer ear. The pulse PPG will use a reflectance method for measurement. Placing the pulse sensor 9 inside the earpiece 3 which covers the ear, reduces signal noise from ambient light. The PPG data is converted into the following biometric signals (but not limited to these): heart rate (pulse), heart rate variability, and pulse volume.

The present invention may be better understood with reference to the following example where outcomes of tPBM stimulation are assessed using cognitive tests with behavioral responses, such as reaction time and accuracy, event-related potentials (ERP), EEG oscillations and event-related EEG oscillations (ERO). Wherein cognitive testing may be completed by a person before, during, or after transcranial photobiomodulation (tPBM).

In one example of a cognitive test protocol the present invention may stimulate brain using tPBM and administer cognitive test with reaction time and accuracy, as well as ERP and EEG oscillation recording at sites Fz, Cz, and Pz considered as most popular topographic sites for ERP analysis. Other locations and/or alternate locations may also be selected. This protocol may be implemented with tPBM stimulation before, during, or after administration of forced-choice neurocognitive tests such as Eriksen flanker test (Eriksen & Eriksen, 1976). Modification of this flanker test uses behavioral motor response such as button press and evaluates reaction time and accuracy, as well as EEG-based assessment of stimulus-locked and response-locked event-related potentials (ERP) and EEG oscillations (ERO).

Flanker tests (Eriksen & Eriksen, 1974) with EEG recording is a task aimed at assessment of attention using ERP methods. The flanker test is one of the focused attention tasks usable for evaluation of executive functions that include cognitive processes such as selective attention, response inhibition, performance monitoring, and working memory. In the flanker task, users typically decide which of several stimuli have been presented in the middle of the string is the target to respond while simultaneously ignoring the stimuli that are presented at the left and right of the central stimulus, so called flankers The flanker tasks in the most popular modification requires spatial selective attention and executive control. In this task, irrelevant flankers must be inhibited in order to respond to a centrally located relevant target stimulus. Incompatible trials with incongruent flankers that are different from the central target stimulus result in slower reaction time and increased number of commission errors. Exercise of attention is required to effectively resolve the interference of flanker stimuli and conflict between competing distracting stimuli and responses during performance in the flanker task.

This test is of especial interest since it can readily produce more commission errors and allows analysis of error processing, monitoring and correcting processes believed to be controlled by the frontal and central cortical areas (Falkenstein et al., 2000; Nieuwenhuis et al., 2001). These areas can be stimulated by transcranial photobiomodulation in the present invention. In the flanker test when commission errors are committed the response-locked ERPs of interest are error-related negativity (ERN) and error-related positivity (Pe). The ERN is a negative-going ERP wave that starts peaking around 50 ms post-error. In one embodiment the ERN is measured during a response inhibition paradigm such as the flanker task, wherein users see a target arrow stimulus within a set of other arrow stimuli flanking on both sides of target arrow showing correct direction to press either left or right button. In some trials of the test the flankers are the same as the target (congruent trials) but in other occasions, the flankers are different from the central target arrow (incongruent trials).

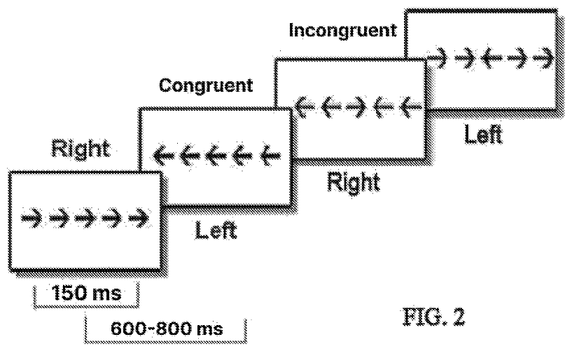

An arrow version of the Eriksen flanker task used widely to elicit the ERN. In some modifications signs like “<” and “>” are used to show direction of response. Example of an arrow flanker test is illustrated in FIG. 2. On each trial, participants view five arrows presented for 150 ms or 200 ms; they are asked to respond as quickly and as accurately as possible to indicate the correct direction of the middle arrow and press the left or right button of the controller. Participants have up to approximately 800 ms-1000 ms from the onset of the stimulus to respond. Half of the trials are congruent (<<<<< or >>>>>), respond left, respond right respectively), whereas the other half are incongruent (e.g., >> < >> or << > <<). Users receive short breaks throughout the task. A longer version of the task can have 360 or even up to 720 trials, while a shorter version of the task is also acceptable but produces less errors.

In yet another embodiment a modification of the flanker test is used which includes A No-Go element in the task. The flanker with Go/NoGo task modification combines the flanker task with a Go/NoGo response paradigm (Ruchsow et al., 2005). In this version the visual stimulus includes four arrows and one non-arrow sign such as for example ‘=” presented centrally for 150 ms or 200 ms. The inter-trial interval is 1000 ms. Left- or right-hand responses are required when the middle target arrow is either a “<” (Go press left button) or a “>” (Go press right button). In addition, there are trials with incongruent NoGo stimulus (<<=<< or >>=>>). These NoGo trials (“=” in the center) require responses to be withheld, producing more commission errors. The number of Go congruent, Go incongruent, and NoGo incongruent trials can be adjusted in this flanker protocol modification.

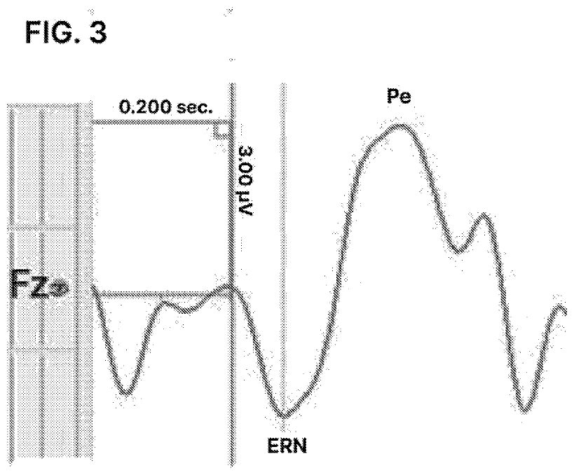

Response-locked ERN and Pe potentials are triggered by committed error response and are reflecting processes related to error detection, error monitoring and those related to error awareness. These error-specific components are the error-related negativity (ERN, more rarely referred to as Ne) and the error-related positivity (Pe). The ERN is a response-locked negative ERP deflection, emerging between 0 and 150 ms after the onset of the incorrect behavioral response—a commission error. The ERN is followed by a positive wave referred to as the Pe potential (100 ms-200 ms range). Waveforms of ERN and Pe stimulus-locked ERP components are illustrated in FIG. 3. The Pe is thought to be related to the conscious recognition of the error or the attribution of motivational significance to the committed error. It is suggested that while the ERN indicates an initial automatic response of error detection, whereas the Pe reflects the conscious comprehension of the error. The ERN/Pe waves are associated with self-monitoring, self-correction and post-error slowing responses, and are interpreted as biomarkers of error processing and committed error awareness (Falkenstein et al., 2000; Nieuwenhuis et al., 2001).

Behavioral response measures in the flanker test may include mean reaction time and response accuracy (percent of correct hits). Number and percent of commission and omission errors are calculated for each test session. Stimulus-locked ERPs in flanker test, including one with NoGo trials in present example of embodiment are posterior (parietal, Pz) N200 ERP and P300 (P3b) in only correct responses to congruent and incongruent stimuli. In flanker test modification with Go-NoGo trials, additional measures of interest include difference wave NoGo-N2 and NoGo-P3 at the Fz site. Both NoGo-N2 and NoGo-P3 are calculated as difference between NoGo-N2 and NoGo-P3 and Go-N2 and Go-P3 at frontal sites (e.g., Fz) within windows typical for N200 (180 ms-320 ms) and P300 (300 ms-500 ms) ERPs. These measures are considered as EEG biomarkers of cortical inhibition processes. They are registered as well in a flanker without NoGo trials.

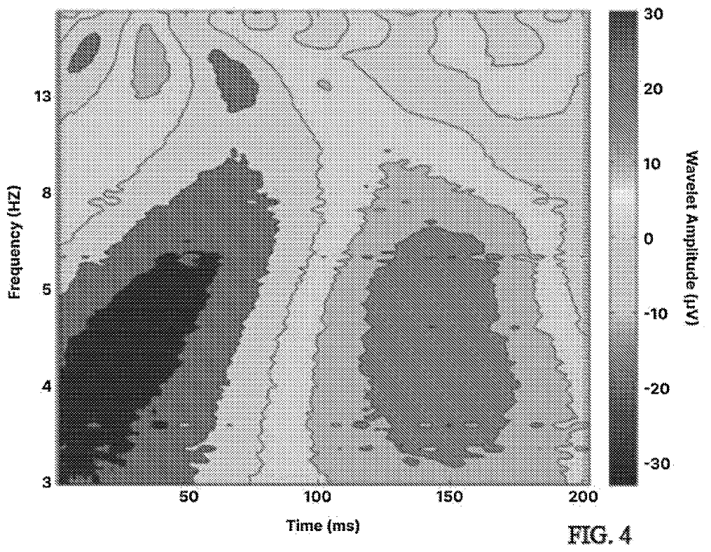

A longer version of the task can have 360 or even 720 trials, while a shorter version of the task is also acceptable but produces less errors. In embodiments of the flanker test when shorter version is used, and number of error trials is low, the method of analysis of error-related EEG responses in yet another embodiment uses single trial time-frequency analysis based on wavelet transformation. Single-trial analysis of EEG activity is important in order to detect and analyze error-related negativity (ERN) in response to commission error. Method of ERN and Pe analysis using wavelet transformation is suggested to be employed in this embodiment. Single trial EEG data from errors in the flanker task in present embodiment may be processed using a continuous wavelet transform. Coefficients from the transform that corresponded to the theta range are averaged to isolate a theta waveform in the time-frequency domain. Measures called the time-frequency ERN and Pe are obtained from these waveforms for midline frontal and central EEG sites (Fz and Cz) for each error trial. A comparison of the amplitude and latency for the time-frequency ERN and Pe in flanker test administered before, during, or after tPBM session or the course of tPBM is performed to assess effect of stimulation on error monitoring and correction function of the user. This single trial time-frequency error analysis method is suitable for examining error processing when a user commits only a few errors (Clemans et al., 2012). Illustration of wavelet-based time frequency analysis of single trial ERN and Pe is presented in FIG. 4.

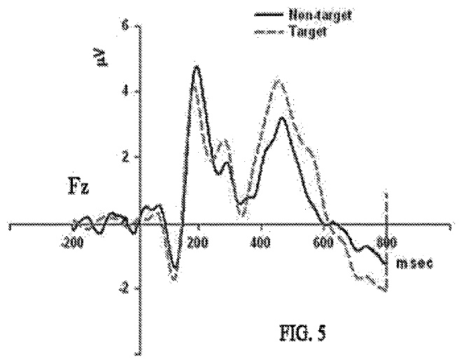

In yet another embodiment of the device and method a visual or auditory oddball test can be used for assessment of cognitive status of the user before, during, or after transcranial photobiomodulation or after the course of tPBM treatment. In visual oddball test example, the ERP test paradigm is used for cognitive processes and attention measurement. The visual oddball paradigm is often used to elicit the P3 (P300) cognitive ERP component (Polich & Herbst, 2007; Herrmann & Knight, 2001). In the traditional visual two-stimulus oddball test, the target stimulus is presented infrequently among frequent standard stimuli. In the three-stimulus oddball test version, the rare target stimulus is presented along with frequent standards and infrequently occurring distractor stimuli (might be the same rare stimulus or several novel distractors). The user must respond to the attended target stimulus and ignore any other stimuli. The target and novel stimuli elicit a large positive P3 (or P300) potentials, specifically with the frontal P3a component at Fz and parietal P3b component at Pz electrodes with a peak latency within 300-400 ms post-stimulus. The parietal P3 (P3b) amplitude is interpreted as an update of the mental representation of a stimulus. In the three-stimulus version of visual oddball test novel distracter stimuli elicit frontal P3a ERP, that is interpreted as a marker of attentional orienting. FIG. 5 illustrates P3a component elicited at the frontal Fz site in response to target and non-target stimuli. Therefore, in the three-stimulus modification of visual oddball test with novel distracters, P300 (P3) potential is further divided into the P3a and P3b subcomponents (Polich, 2007). The P3a elicited by an infrequent and uninstructed novel stimulus is localized in the frontal (e.g., Fz) or central (Cz) cortices and has relatively short latency as compared to the P3b component that is elicited in response to attended infrequent stimulus and is localized in the parietal area (e.g., Pz).

The P3a component reflects processes related to the selection of stimulus associated with attentional orienting. The amplitude of the P3a reflects processes indicating focal attention. The P3b is reflecting processes related to the allocation of attentional resources during performance in cognitive tasks and is associated with updating working memory. The P3b amplitude is reflecting the attentional resources allocated to processing a stimulus, whereas the P3b latency reflects stimulus classification speed. Target detection in the visual oddball paradigm described above is also associated with a late response at parietal areas (e.g., Pz), starting at 200 ms and continuing for up to 500 ms, including the negative N2 (N200) and positive P3b components. Both P3a and P3b are analyzed to calculate peak amplitude and latency of the peak within preselected window, though in some cases instead of max peak, mean value of component or the area (magnitude is calculated) and in more rare cases N2-to-P3b peak-to-peak amplitude is used.

In a most simple modification, two visual stimuli, for example letters “O” and “X” are designed as the standard and target stimuli, respectively. The users are instructed to press “X” for a target stimulus and not to respond for a standard stimulus. Further, the reaction time and correct target detection of the user are recorded. Two types of error are expected: commission error-“false alarm” (i.e., pressed key when standard stimulus was shown, reflects impulsivity) and omission error (forgot to press key when target stimulus appeared, reflects inattention).

In yet another example of the embodiment the three-stimulus visual oddball with novel distractors test may be used. This ERP test as stimuli uses letters “X,” “O,” and novel distracters (“v,” “\hat{ }”, “>,” and “<” signs). One of the stimuli (“O”) is presented on 80% of the trials (frequent standard); the novel stimuli stimulus (e.g., “>”) is presented on 10% (2.5% for each of signs) of the trials (rare distracter), whereas the third (“X”) is presented on the remaining 10% of the trials and represents the target. Users are instructed to press a button when they see the target letter on the screen. Event-related potentials (ERP) locked to stimulus events (triggered by target) reflect the activation of neural structures in primary sensory cortex, and in associative cortical areas related to higher order cognitive processes. The ERP analysis provides temporal information concerning processes such as attention. Earlier ERP components, such as the P100, N100, P200, usually relate to early attentional selection mechanisms, whereas later components (N200, P300/P3b) are more often associated with organization and interpretation of the stimulus.

The negative ERP (N200 located over centro-parietal sites occurs within 180 and 320 ms window post-stimulus. This component is believed to be associated with categorization, perceptual closure and attention focusing and signaling about formation of a perceptual representation. The visual N200 is larger if the stimulus contains a perceptual feature or attribute that defines the target to be attended in the test. In a three-stimulus oddball task the P3a is interpreted as orienting response to novel distractions, while the P3b is considered as an index of sustained attention to target.

In another embodiment of the device and the methods, the system analysis of EEG responses uses time-frequency wavelet-based analysis of EEG in single trials and more specifically EEG evoked and induced gamma (35-45 Hz or 30-80 Hz) oscillations and EEG frequencies (e.g., theta-4-8 Hz and gamma-35-45 Hz) phase-amplitude coupling methods. Wherein cognitive testing may be completed by a person before, during, or after transcranial photobiomodulation (tPBM).

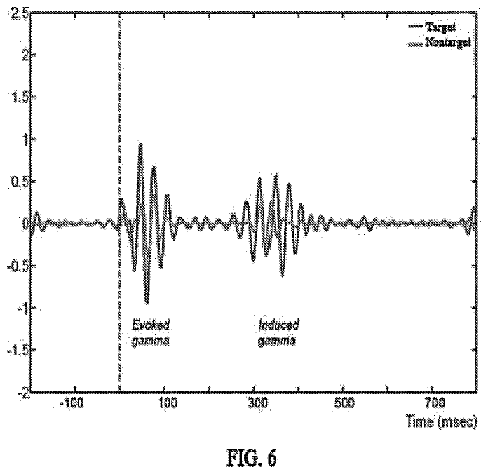

In another embodiment of the cognitive tests in present invention wavelet-based time-frequency analysis of event-related EEG gamma oscillations is used. During processing of collected EEG data, the oscillatory response of the 35-45 Hz centered gamma band or wider gamma range (e.g., 30-80 Hz) is broken down into two main groups: evoked and induced responses. These two gamma responses are discriminated based on temporal localization and if they are time-locked to a stimulus. Event-related oscillations are divided into “evoked” and “induced” components depending on their relationship to the event, i.e., stimulus. The early, or so called “evoked” gamma responses occur in the 40-180 ms post-stimulus range. These evoked responses have been attributed to the early information processing linked to the sensory processes and early stages of stimuli perception (Basar, 2013). They are closely time-locked to a specific stimulus. On the other hand, the later occurring induced gamma oscillation responses are better manifested within the post-stimulus window of 250-500 ms range. FIG. 6 illustrates evoked and induced gamma oscillations in 35-45 Hz range waveforms in response to target and non-target stimuli during cognitive test. The induced gamma oscillation responses are observed in tasks requiring higher-order processes of the short-term memory (Herrmann et al., 2014). Event-related gamma oscillations have been associated with indication of perceptual and cognitive processes and considered to be representing an integration of attentional resources and cognitive processes.

Previous studies revealed that evoked and induced responses reflect different neural processes and mechanisms (Basar, 2013; Herrmann & Demiralp, 2005; Tallon-Baudry & Bertrand, 1999). It was reported in the art that induced gamma oscillation power is increased at around 40 Hz during a short-term memory task and is related to maintaining an object representation in short-term memory (Tallon-Baudry & Bertrand, 1999). In present invention application of single trial evoked and induced gamma oscillations power during cognitive tests such as flanker test or visual or auditory oddball tests with EEG recording are used to assess and evaluate sensory and cognitive process of the user of the device before, during, or after transcranial photobiomodulation session or after the course of tPBM or biofeedback treatment.

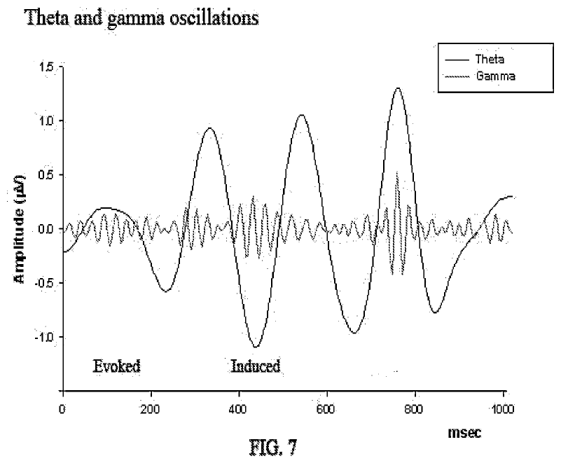

In yet one more embodiment of the present invention the method of cognitive testing examines attentional processes operating in early pre-attentive sensory processes, such as initial orienting, in sustained attention by time-frequency measures of EEG oscillations of several EEG bands during performance on task. Of particular interest in this regard are theta (4-8 Hz) and 40 Hz-centered gamma oscillations. FIG. 7 illustrates phase-amplitude coupling of theta and gamma oscillations during evoked and induced oscillations in response to stimulus in a cognitive test. Previous research suggests that theta oscillations are indicative of neural processes involved in the integration of percepted stimuli in following working memory, attention, and long-term memory processes (Davoudi et al., 2021; Lisman & Jensen, 2013).

In is known to those skilled in the art that EEG oscillations exhibit phase-amplitude coupling in certain physiological states or during performance of specific tasks and research on neural oscillations suggests that the interaction between the brain regions is processed by a cross-frequency coupling between low-frequency band phase and high frequency band amplitude. In particular, the cross-frequency coupling between the theta (4-8 Hz) phase and the gamma (predominantly in 40 Hz centered range, e.g., 35-45 Hz) amplitude may play an important functional role in cognitive activities such as attention and working memory (Canolty et al., 2006). Specifically, EEG responses to visual stimuli are known to be marked by readily observed changes in theta and gamma oscillations. Cross-frequency coupling method in the present method measures the association between the theta oscillation phase and the gamma power. Higher-magnitude theta-gamma coupling values translate into greater gamma amplitude during the theta phase (Lisman & Jensen, 2013). Theta-gamma coupling has been shown to be a functionally important functional role for processes related to short-term and long-term memory. Research suggests that phase-amplitude coupling between the theta phase and gamma amplitudes represents cognitive control mechanisms (Köster et al., 2014). In present embodiment event-related evoked and induced theta and gamma EEG oscillations are analyzed for calculation of theta and gamma activity phase-amplitude coupling for evaluation of cognitive processes of user in response to effects produced by transcranial photobiomodulation or biofeedback.

Cognitive testing in various embodiments of the present invention may include memory testing, physical reaction testing, audio processing testing, visual processing testing, emotional response testing, attention testing, executive function testing, learning testing, reaction time testing, peripheral vision testing, intelligence testing and language testing. During the cognitive testing, one or more of ERP, ERO, EEG and HRV data can being evaluated.

In one embodiment, the present invention utilizes a cognitive assessment wherein the subject of the assessment executes cognitive assessment trials and then receives a dose of tPBM stimulation, followed by additional cognitive assessment trials. The sensors of the present invention are used to collect biometric signals from the subject during the cognitive assessment and tPBM stimulation including (but not limited to these): EEG (electroencephalography), heart rate, pulse volume, heart rate variability (HRV), and other physiological measures.

In one variation the pre-tPBM trials, tPBM stimulation, and post-tPBM trial phases may be executed immediately one after the other; while in yet another variation, breaks may be provided to the subject between one or more of these phases. In another variation the tPBM stimulation may be provided to the subject during some or all of the cognitive assessment trials.

In one embodiment differences in the ERP components before and after the tPBM stimulation may be evaluated including but not limited to P300, P3a, P3b, N200, ERN, Pe, reaction time, reaction time after error, No-Go N2, No-Go P3. Further comparative measures based on pre-tPMB trials, active-tPMB trials, and post-tPBM trials may include difference waves, timing, amplitude, and other measures.

In yet another embodiment, cognitive assessment trials may be analyzed for evoked and induced gamma oscillations power, timing, amplitude and cross-frequency coupling between the theta (4-8 Hz) phase and gamma (35-45 Hz) and other EEG measures. Wherein comparative measures can be calculated to evaluate differences in timing, amplitude, and frequency of pre-tPMB trials, active-tPMB trials, and post-tPBM trials. In present invention application of single trial evoked and induced gamma oscillations power during cognitive tests such as flanker test or visual or auditory oddball tests with EEG recording are used to assess and evaluate sensory and cognitive process of the user of the device before, during, or after transcranial photobiomodulation session or after the course of tPBM or biofeedback treatment.

One embodiment of the present invention cognitive assessment trials include the user executing memory based tests. Wherein the user is presented with an image, sound, letters, and or numbers to remember for a set period of time. After another period of time passes during which the user may be distracted by performing another task, the user is asked to identify the remembered item. In this embodiment performance measures may be compared between pre-tPMB trials, active-tPMB trials, and post-tPBM trials. This embodiment may be combined with analysis of EEG oscillations.

In the present invention the measures from cognitive testing with tPBM may be utilized in the evaluation of cognitive function and or diagnostics of cognitive conditions such as but not limited to mild cognitive impairment (MCI), Dementia, Alzheimers, Autism Spectrum Disorder (ASD), Obsessive Compulsive Disorder (OCD), Anxiety, ADD and ADHD. Wherein the measures may be compared to statistical norms, calibrated algorithms, or used in artificial intelligence networks.

In another embodiment, a close loop adaptation of tPBM stimulation and cognitive assessment is performed over the course of one or more treatment sessions and one or more assessment trials in order to optimize results. Wherein the user's response to tPBM stimulation is measured and adapted through cognitive assessment trials. Wherein tPBM adaptation is included but is not limited to frequency, timing, duration, duty cycle, location, dose. Wherein adaptations are selected by the system in order to optimize the cognitive performance and measures of the subject.

Alternative embodiments included, but are not in any way limited to, integration of the neurofeedback and neurostimulation modalities of the present invention into other suitable wearable devices besides headphones. One skilled in the art will understand that such alternative embodiments are possible as long as stimulation and measurement is possible to the appropriate regions of a wearer's brain. One skilled in the art will also understand that application of other known PBM, EEG and HRV biofeedback and ERP and EEG oscillations (ERO) test protocols would be possible within the scope of the current invention. The working examples provided herein are illustrative in nature and are not intended to limit the scope of the disclosure.

The above-described embodiments should be considered as examples of the present invention, rather than as limiting the scope of the invention. In addition to the foregoing embodiments of the invention, review of the detailed description and accompanying drawings will show that there are other embodiments of the present invention. Accordingly, many combinations, permutations, variations and modifications of the foregoing embodiments of the present invention not set forth explicitly herein will nevertheless fall within the scope of the present invention.

### REFERENCES CITED

### Patent Documents

10,987,521 B1 April 2021 Chicchi
2009/0254154 A1 October 2009 De Taboada and Streeter
2010/0204762 A1 August 2010 De Taboada and Streeter
2019/0335551 A1 October 2019 Williams et al.
2021/0001147 A1 January 2021 Huang
2021/0016103 A1 January 2021 Huang
2021/0023391 A1 January 2021 Huang
U.S. Pat. No. 9,675,292 B2 July 2017 Fadem
WO 2020/223397 A1 November 2020 Mclaughlin
WO 2019/053625 A1 March 2019 Lim
EP 2260760 B1 August 2014 Fadem
AU 2015388475 B2 September 2016 Lim

### OTHER PUBLICATIONS

Başar, E. (2013). A review of gamma oscillations in healthy subjects and in cognitive impairment. International Journal of Psychophysiology, 90 (2), 99-117. https://doi.org/10.1016/j.ijpsycho.2013.07.005
Berman, M., & Nichols, T. (2019) Treatment of Neurodegeneration: Integrating Photobiomodulation and Neurofeedback in Alzheimer's Dementia and Parkinson's: A Review. Photobiomodulation, Photomedicine, and Laser Surgery, 37, 10, https://doi.org/10.1089/photob.2019.4685
Blanco, N. J., Saucedo, C. L., & Gonzalez-Lima, F. (2017). Transcranial infrared laser stimulation improves rule-based, but not information-integration, category learning in humans. Neurobiology of Learning and Memory, 139, 69-75. 10.1016/j.nlm.2016.12.016
Canolty, R. T., Edwards, E., Dalal, S. S., Soltani, M., Nagarajan, S. S., Kirsch, H. E., Berger, M. S., Barbaro, N. M., & Knight, R. T. (2006). High gamma power is phase-locked to theta oscillations in human neocortex. Science, 313 (5793), 1626-1628. https://doi.org/10.1126/science.1128115
Cardoso, F., Gonzalez-Lima, F., & Gomes da Silva, S. (2021). Photobiomodulation for the aging brain. Ageing Research Reviews, 70, 101415. https://doi.org/10.1016/j.arr.2021.101415
Clemans, Z. A., El-Baz, A. S., Hollifield, M., & Sokhadze, E. M. (2012) Single trial time-frequency domain analysis of error processing in PTSD. Neuroscience Letters. 525 (2), 105-110.
Davoudi, S., Ahmadi, A., & Daliri, M. R. (2021). Frequency-amplitude coupling: A new approach for decoding of attended features in covert visual attention task. Neural Computing and Applications. 10.1007/s00521-020-05222-w.
De la Torre, J. C., Olmo, A. D., & Valles, S. (2020). Can mild cognitive impairment be stabilized by showering brain mitochondria with laser photons? Neuropharmacology, 171, 107841. https://doi.org/10.1016/j.neoropharm.2019.107841
El Khoury, H., Mitrofanis, J., & Henderson, L. A. (2019). Exploring the effects of near infrared light on resting and evoked brain activity in humans using magnetic resonance imaging. Neuroscience, 422, 161-171. https://doi org/10.1016/neuroscience.2019.10.037
Eriksen, B. A., & Eriksen, C. W. (1974). Effects of noise letters upon the identification of a target letter in a nonsearch task. Perception & Psychophysics, 16 (1), 143-149. https://doi.org/10.3758/BF03203267
Falkenstein, M., Hoormann, J., Christ, S., & Hohnsbein, J. (2000). ERP components on reaction errors and their functional significance: a tutorial. Biological psychology, 51 (2-3), 87-107. https://doi.org/10.1016/s0301-0511(99)00031-9
Gonzalez-Lima, F., & Barrett, D. W. (2014). Augmentation of cognitive brain functions with transcranial lasers. Frontiers in systems neuroscience, 8, 36. https://doi.org/10.3389/fnsys.2014.00036
Hamblin M. R. (2016). Shining light on the head: Photobiomodulation for brain disorders. BBA Clinical, 6, 113-124. https://doi.org/10.1016/j.bbacli.2016.09.002
Herrmann, C. S., & Knight, R. T. (2001). Mechanisms of human attention: event related potentials and oscillations. Neuroscience and Biobehavioural Reviews, 25 (6), 465-476. http://dx.doi.org/10.1016/S0149-7634 (01) 00027-6
Herrmann, C. S., Rach, S., Vosskuhl, J., & Strüber, D. (2014). Time-frequency analysis of event-related potentials: A brief tutorial. Brain Topography, 27 (4), 438-450. https://doi org/10.1007/s10548-013-0327-5
Köster, M., Friese, U., Schöne, B., Trujillo-Barreto, N., & Gruber, T. (2014). Theta-gamma coupling during episodic retrieval in the human EEG. Brain Research, 1577, 57-68. https://doi.org/10.1016/j.brainres.2014.06.028
Lapchak, P. A., & De Taboada, L. (2010). Transcranial near infrared laser treatment (NILT) increases cortical adenosine-5′-triphosphate (ATP) content following embolic strokes in rabbits. Brain research, 1306, 100-105. https://doi.org/10.1016/j.brainres.2009.10.022
Lehrer, P. M., & Gevirtz, R. (2014). Heart rate variability biofeedback: how and why does it work?. Frontiers in psychology, 5, 756. https://doi.org/10.3389/fpsyg.2014.00756 Lisman, J. E., & Jensen, O. (2013). The θ-γ neural code. Neuron, 77 (6), 1002-1016. https://doi.org/10.1016/j.neuron.2013.03.007
Luck, S. J. (2014). Event-related potential technique, 2^{nd} ed. MIT. Nieuwenhuis, S., Ridderinkhof, K. R., Blom, J., Band, G. P., & Kok, A. (2001). Error-related brain potentials are differentially related to awareness of response errors: evidence from an antisaccade task. Psychophysiology, 38 (5), 752-760. http://dx.doi.org/10.1111/1469-8986.3850752
Polich, J., & Herbst, K. L. (2000). P300 as a clinical assay: rationale, evaluation, and findings. International journal of psychophysiology, 38 (1), 3-19. https://doi.org/10.1016/0167-8760(00)00127-6
Polich J. (2007). Updating P300: an integrative theory of P3a and P3b. Clinical neurophysiology 118 (10), 2128-2148. https://doi.org/10.1016/j.clinph.2007.04.019
Ramezani, F., Neshasteh-Riz, A., Ghadaksaz, A., Fazeli, S. M., Janzadeh, A., & Hamblin, M. R. (2021). Mechanistic aspects of photobiomodulation therapy in the nervous system. Lasers in medical science, 10.1007/s10103-021-03277-2
Ruchsow, M., Spitzer, M., Groen, G., Grothe, J., & Kiefer, M. (2005). Error processing and impulsiveness in normals: evidence from event-related potentials. Cognitive Brain Research, 24 (2), 317-325. http://dx.doi.org/10.1016/j.cogbrainres.2005.02.003 Salehpour, F., Mahmoudi, J., Kamari, F., Sadigh-Eteghad, S., Rasta, S. H., & Hamblin, M. R. (2018). Brain Photobiomodulation Therapy: a Narrative Review. Molecular neurobiology, 55 (8), 6601-6636. https://doi.org/10.1007/s12035-017-0852-4
Saltmarche, A. E., Naeser, M. A., Ho, K. F., Hamblin, M. R., & Lim, L. (2017). Significant improvement in cognition in mild to moderately severe dementia cases treated with transcranial plus intranasal photobiomodulation: Case series report. Photomedicine and Laser Surgery, 35(8), 432-441. https://doi.org/10.1089/pho.2016.4227
Saucedo, C. L., Courtois, E. C., Wade, Z. S., Kelley, M. N., Kheradbin, N., Barrett, D. W., & Gonzalez-Lima, F. (2021). Transcranial laser stimulation: Mitochondrial and cerebrovascular effects in younger and older healthy adults. Brain Stimulation, 14(2), 440-449. https://doi.org/10.1016/j.brs.2021.02.011
Shaffer, F., & Ginsberg, J. P. (2017). An Overview of Heart Rate Variability Metrics and Norms. Frontiers in public health, 5, 258. https://doi.org/10.3389/fpubh.2017.00258
Sherlin, L., Arns, M., Lubar, J., Heinrich, H., Kerson, C., Strehl, U., & Sterman, B. (2011). Neurofeedback and basic learning theory: Implications for research and practice. Journal of Neurotherapy, 15 (4), 292-304. https://doi.org/10.1080/10874208.2011.623089
Song, P., Han, T., Lin, H., Li, S., Huang, Q., Dai, X., Wang, R., & Wang, Y. (2020). Transcranial near-infrared stimulation may increase cortical excitability recorded in humans. Brain Research Bulletin, 155, 155-158. https://doi.org/10.1016/j.brainresbull.201912.007
Tallon-Baudry, C., & Bertrand, O. (1999). Oscillatory gamma activity in humans and its role in object representation. Trends in cognitive sciences, 3 (4), 151-162. https://doi.org/10.1016/s1364-6613 (99) 01299-1
Tan, G., Shaffer, F., Lyle, R., & Teo, I. (2017). Evidence-based practice in biofeedback and neurofeedback (3rd ed.). Wheat Ridge, CO: Association for Applied Psychophysiology and Biofeedback.
Uozumi, Y., Nawashiro, H., Sato, S., Kawauchi, S., Shima, K., & Kikuchi, M. (2010). Targeted increase in cerebral blood flow by transcranial near-infrared laser irradiation. Lasers in surgery and medicine, 42(6), 566-576. https://doi.org/10.1002/lsm.20938
Vargas, E., Barrett, D. W., Saucedo, C. L., Huang, L. D., Abraham, J. A., Tanaka, H., Haley, A. P., & Gonzalez-Lima, F. (2017). Beneficial neurocognitive effects of transcranial laser in older adults. Lasers in Medical Science, 32 (5), 1153-1162. https://doi.org/10.1007/810103-017-2221-v
Wang, X., Reddy, D. D., Nalawade, S. S., Pal, S., Gonzalez-Lima, F., & Liu, H. (2018). Impact of heat on metabolic and hemodynamic changes in transcranial infrared laser stimulation measured by broadband near-infrared spectroscopy. Neurophotonics, 5(1), 011004. https://doi.org/10.1117/1.NPh.5.1.011004
Wang, X., Dmochowski, J. P., Zeng, L., Kallioniemi, E., Husain, M., Gonzalez-Lima, F., & Liu, H. (2019). Transcranial photobiomodulation with 1064-nm laser modulates brain electroencephalogram rhythms. Neurophotonics, 6(2), 025013. https://doi.org/10.1117/1.NPh.6.2.025013

What is claimed is:

1. A wearable device for stimulating and monitoring the brain comprising:
at least one transcranial PBM stimulator; and
at least one EEG sensor;
wherein the device is configured to provide stimulation to a brain of a human from the transcranial PBM stimulator to change electrical activity of the brain of the human;
and wherein the device is further configured to monitor the electrical activity using the EEG sensor;
and wherein the device is further configured so that changes in electrical activity detected using the EEG sensor can be evaluated using cognitive testing.

2. The wearable device of claim 1, wherein the device is further configured to receive results of cognitive testing and, on the basis of the results, provide further stimulation to the brain of the human from the transcranial PBM stimulator to influence subsequent cognitive test results.

3. The wearable device of claim 2, further comprising at least one additional sensory stimulator for providing visual or auditory stimulation, and wherein the device is further configured to, on the basis of the results and EEG sensor data, provide further stimulation to the brain of the human from the additional sensory stimulator.

4. The wearable device of claim 3, wherein cognitive testing may be selected from the group consisting of memory testing, physical reaction testing, audio processing testing, visual processing testing, emotional response testing, attention testing, executive function testing, learning testing and language testing, and wherein during the cognitive testing, one or more of ERP, ERO, EEG and HRV data is being evaluated.

5. The wearable device of claim 4, wherein the further stimulation from the transcranial PBM stimulator and the additional sensory stimulator is tailored to influence one or more of ERP, ERO, EEG and HRV.

6. The wearable device of claim 2, further comprising at least one additional biometric sensor configured to provide recording of biometric data of the wearable device to monitor responses during the PBM stimulation and cognitive testing.

7. The wearable device of claim 6, wherein the at least one additional biometric sensor is a heart rate sensor that uses a photoplethysmogram (PPG) or a pulse oximeter sensor or an ECG sensor or a GSR sensor.

8. The wearable device of claim 2, wherein the at least one transcranial PBM stimulator may be a near-infrared laser or an LED that emits light at a wavelength of between 600-1150 nm.

9. The wearable device of claim 2, comprising at least two transcranial PBM stimulators that may be pulsed at different frequencies.

10. The wearable device of claim 2, wherein the at least one EEG sensor is configured to obtain data on electrical activity of the brain during cognitive testing simultaneously to the at least one transcranial PBM stimulator providing pulsed near-infrared light to the brain.

11. The wearable device of claim 6 wherein the at least one EEG sensor and the at least one additional biometric sensor are configured to obtain biometric data during cognitive testing simultaneously to the at least one transcranial PBM stimulator providing pulsed near-infrared light to the brain.

12. The wearable device of claim 2, wherein the wearable device takes the form of a head mounted interface.

13. The wearable device of claim 12, wherein the wearable device takes the form of a set of head mounted headphones.

14. The wearable device of any of the previous claims, wherein the device is further configured to, on the basis of cognitive testing results, provide further stimulation to the brain of the human over time and evaluate resulting changes in cognitive testing results.

15. The wearable device of any of the previous claims, wherein the device is further configured to provide stimulation to the brain of the human from the transcranial PBM stimulator and evaluate the results of cognitive testing in order to evaluate or diagnose cognitive function or cognitive conditions.

16. A method of optimizing the biometric parameters of a human comprising:
providing the human with a wearable device for stimulating and monitoring the brain comprising:
at least one transcranial PBM stimulator; and
at least one EEG sensor;
wherein the device is configured to provide stimulation to a brain of a human from the transcranial PBM stimulator to change electrical activity of the brain of the human;
and wherein the device is further configured to monitor the electrical activity using the EEG sensor;
and wherein the device is further configured so that changes in electrical activity detected using the EEG sensor can be evaluated using cognitive testing;
and wherein the device is further configured to receive results of cognitive testing and, on the basis of the results, provide further stimulation to the brain of the human from the transcranial PBM stimulator to influence subsequent cognitive test results;
administering cognitive testing to the human;
measuring one or more of the human's EEG, ERP, ERO and HRV data during cognitive testing;
providing transcranial PBM stimulation to the human before, during or after the cognitive testing; and
on the basis of the results of cognitive testing, administering transcranial tPBM to optimize the human's EEG data or ERP data.

17. The method of claim 16, wherein measuring the ERP's of the human comprises evaluating one or more of P300, P3a, P3b, N200, ERN, Pe, reaction time, reaction time after error, No-Go N2 and No-Go P3.

18. The method of claim 16, wherein measuring the EEG data of the human comprises evaluating EEG oscillations of one or more EEG bands comprising theta and gamma bands.

19. The method of claim 16 further comprising measuring at least one additional biometric parameter before, during, and after PBM stimulation and providing biofeedback to the wearer.

20. The method of claim 16, further comprising providing audio or visual stimulation to the human in addition to PBM stimulation to further optimize feedback of the human's biometric parameters.

21. The method of claim 16, wherein the at least one other biometric sensor may be a heart rate sensor that uses a photoplethysmogram (PPG) or a pulse oximeter sensor or an ECG sensor or a GSR sensor.

22. The method of claim 16, wherein the at least one PBM stimulator may be a near-infrared laser or an LED that emits light at a wavelength of between 600-1150 nm and may be pulsed at different frequencies.

23. The method of claim 16, wherein the at least one EEG sensor may obtain data on electrical activity of the brain during cognitive test simultaneously to the at least one PBM stimulator providing pulsed near-infrared light to the brain.

24. The method of claim 16, wherein the at least one EEG sensor and the at least one additional biometric sensor provide biometric data that may be used in the assessment of results of the cognitive testing simultaneously to operation of the PBM stimulator.
